(19) United States
(12) Patent Application Publication
(10) Pub. No.: US 2005/0020896 A1
(43) Pub. Date: Jan. 27, 2005

(54) Apparatus and method of intraocular pressure determination

(76) Inventors: Terry A. Fuller, Jenkintown, PA (US); Francis E. O'Donnell JR., Town & Country, MO (US); Yongping Wang, Philadelphia, PA (US)

Correspondence Address: Paul M. Denk;Ste. 170, 763 S. New Ballas Road, St. Louis, MO, 63141, US

(21) Appl. No.: 10/890,615

(22) Filed: Jul. 14, 2004

Related U.S. Application Data
(60) Provisional application No. 60/489,681, filed on Jul. 24, 2003.

(51) Int. Cl. A61B 3/16; A61B 8/00

(52) U.S. Cl. 600/405; 600/449

(57) ABSTRACT

An improved apparatus and method of intraocular pressure determination is disclosed in which applanation tonometery is done simultaneously with pachymetry. The method allows for increased accuracy of intraocular pressure determination based upon adjustments of applanation tonometry for corneal thickness. The device allows an untrained operator to quickly and easily determine the accurate intraocular pressure.

### CROSS REFERENCE TO RELATED APPLICATION

This non provisional patent application claims priority to the provisional patent application having Ser. No. 60/489,681, which was filed on Jul. 24, 2003.

### FIELD OF THE INVENTION

The present invention is a novel applanation tonometer used to accurately measure intraocular pressures for the purpose of diagnosing and monitoring treatment for glaucoma and ocular hypertension. Specifically, the applanation is done with both an ultrasonic transducer that measures corneal thickness at the point of applanation and an intraocular pressure (IOP) measuring transducer. Since applanation pressure is a function of corneal thickness, the simultaneous determination of both variables at the same location allows for more accurate determination of intraocular pressure. The configuration of the applanation IOP measuring transducer and the ultrasonic transducer allows a minimally trained operator to determine the precise endpoint of applanation.

### BACKGROUND OF THE INVENTION

Glaucoma refers to a specific pattern of optic nerve damage and visual field loss caused by a number of different eye diseases. Frequently, these diseases are characterized by elevated intraocular pressure; a leading risk factor for development of glaucoma. Millions of people worldwide suffer from glaucoma, at least half of which do not know they have the disease because glaucoma has no symptoms until there is generally irreversible vision loss. Devices that measure intraocular pressure are referred to as tonometers.

Applanation tonometery was popularized by Goldmann as an improved method of intraocular pressure determination in comparison to indentation tonometery or invasive intraocular pressure measurements. Goldmann applanation tonometery uses and indirect pressure measurement technique based on the Imbert-Fink principal which teaches that pressure inside a liquid filled sphere can be determined by measuring the force required to flatten a portion of the surface. There are several indirect measurement devices in addition to the Goldmann tonometer that have been conceived, e.g. the Mackey Marg, Perkins and Draeger to name a few. They measure either the degree of indentation of the cornea produced by an application probe or they measure the force required for the probe to flatten a defined area of the cornea. Details of such previous devices are widely available in numerous textbooks and will not be discussed herein.

It will be obvious to one knowledgeable in the art that variations in thickness of the cornea would affect the accuracy of its applanation in the indirect measuring techniques. Specifically, a thinner than normal cornea would applanation easier than a normal thickness cornea, thereby generating a falsely low measure of intraocular pressure. Conversely, a thicker than normal cornea would overestimate the true intraocular pressure. Since the diagnosis of glaucoma and the assessment of the adequacy of treatment are largely dependent upon intraocular pressure, the accuracy of intraocular pressure measurements is of paramount importance. Presently, in order to determine variations in corneal thickness, prior art has used pachymetry by optical or ultrasonic means to measure corneal thickness. It is time-consuming and expensive to use a second instrument, e.g. an ultrasonic pachymeter, sequentially with the tonometer. Moreover, it is impossible to know if the portion of the cornea applanated for tonometery was the portion whose thickness was measured. Further, the determination of both applanation tonometery and corneal pachymetry requires solving and equation in order to calculate the true intraocular pressure. As a result, the correction of applanation tonometery for corneal thickness variables is generally not widely done except in academic or research circumstances.

Recently, studies of ocular hypertensive patients sponsored by the National Eye Institute (NEI) of the National Institutes of Health (NIH) have demonstrated that corneal thickness is the single most important predictor of glaucoma. Corneal thickness is inversely proportional to the risk of developing glaucomatous damage. That is to say, among ocular hypertensives, the thinner the cornea the greater the risk of glaucoma.

During Goldmann applanation tonometery, the so-called “gold standard” for measurements of intraocular pressure, a fluorescent dye is applied to the corneal surface to aid in the pressure measurement. In an upright patient, the operator looks through ocular is of a slit lamp microscope in order to obtain a clear view of the cornea through the applanation device. Under direct vision of the operator, the applanation element is momentarily pressed on to the cornea by the operator. The cornea flattens as a result of the force applied by the applanation element. This in turn causes a change in the pattern of fluorescence. The operator observes these changes and when the pattern of fluorescence reaches a predetermined endpoint the intraocular pressure is determined. This method also helps to reduce inadvertent trauma to the delicate epithelial layer of the cornea. This technique as well as measurements with the classical tonometers requires training, skill and experience. Technique is critical with present tonometery because it is important not to under applanate or over applanate the cornea. A well-trained and skilled operator is required in order to obtain accurate and repeatable results.

In the U.S. Pat. No. 6,083,161 and as taught in CIP Ser. No. 10/234,294, filed on Sep. 3, 2002, O'Donnell disclosed new apparatus and method which provides more accurate intraocular pressure determination. The apparatus measures conventional tonometery as well as corneal thickness using a single integrated device. Both measurements are made on the exact region of the cornea. The apparatus uses a transparent corneal applanation element for use in the determination of the applanation pressure. An ultrasonic transducer is preferably coaxial with or part of the tonometer transducer and is used to measure corneal thickness. Such a design would normally partially skewer the view of the cornea and make the measurement difficult or impossible. However the improved apparatus uses an internal reflection technique in order to view around the obscuration. However, this improved method still suffers from the difficulty of measurement as described above including use of fluorescein dye viewed through an expensive and generally non-mobile slit-lamp microscope in patients seated in an upright position. Further, it requires a well-trained and skilled operator in order to obtain accurate and repeatable results.

Hyman teaches a method for determining intraocular pressure using a conventional slit lamp-based Goldmann style tonometer and a pachymeter correcting for corneal thickness. After the pachymeter signal is generated, this method requires the application probe to be moved in a direction toward the subjects' eye until a measurement endpoint is observed by the observer. This method is cumbersome and costly. In addition, the method requires the application probe to be in contact with the cornea for a long time; sufficient to change between the two sensors. Contact with the cornea for an extended period of time can alter the intraocular pressure and is uncomfortable for the patient.

There are instances where accurate IOP determination is required and where skilled operators are not present, e.g. examining patients during hospital rounds, emergency rooms, private ophthalmic and optometrist's offices, intern's offices, etc. Further, the use of a portable or handheld tonometer is beneficial or required when the patient is not in an upper right position, e.g. the operating room during surgery, use with children and infants and during patient rounds on the hospital floors. While there are portable tonometers available they cannot measure or correct for corneal thickness.

Other prior art showing related technology can be seen in the following patents.

| Patent | Pub. Date | Inventor | Assignee | Title |
|---|---|---|---|---|
| 4,930,512 | Jun. 5, 1990 | Henriksen | Sonomed, Inc. | Hand held spring-loaded |
| | | | | ultrasonic probe |
| 5,165,415 | Nov. 24, 1992 | Wallace | Bio-Rad Labs, | Self contained hand held |
| | | | Inc. | ultrasonic instrument for |
| | | | | ophthalmic use |
| 5,355,884 | Oct. 18, 1994 | Bennett | | Applanation Tonometer for |
| | | | | measuring intraocular pressure |
| 5,389,848 | Feb. 14, 1995 | Trzaskos | General Electric, | Hybrid ultrasonic transducer |
| | | | Co. | |
| 5,474,066 | Dec. 12, 1995 | Grolman | Leica, Inc. | Non-contact tonometer |
| 5,636,635 | June 1997 | Massie et al. | Massie Research | Non-contact tonometer |
| | | | Laboratories, Inc. | |
| 6,083,161 | Jul. 4, 2000 | O'Donnell | Sublase, LLC | Apparatus and method for |
| | | | | improved intraocular pressure |
| | | | | determination |
| 6,113,542 | Sep. 5, 2000 | Hyman | | Diagnostic apparatus and method |
| | | | | to provide effective intraocular |
| | | | | pressure based on measuring |
| | | | | thickness of the cornea |

### SUMMARY OF THE INVENTION

There exists a need, therefore, for a simple to use, portable device that does not require trained personnel to simultaneously perform tonometery and pachymetry, register more accurate intraocular pressure for general clinical use and be suitable for use in any position. The present invention applanates the cornea with an ultrasonic transducer while simultaneously recording applanation pressure and corneal thickness in the exact region of applanation. The present invention can be configured for use as either a fixed or mobile device and can be used in any position. A microprocessor converts the applanation pressure to an adjusted intraocular pressure which more accurately reflects the true intraocular pressure when compared to conventional applanation tonometery. This device and method allows for quick, convenient, easy to use, portable and precise determination of intraocular pressure.

It is an object of the present invention to provide a device which can easily and accurately determine intraocular pressure regardless of variations in corneal thickness.

It is a further objective of the present invention to provide pachymetry determination in the exact region of cornea applanation IOP measurements.

It is a further object of the present invention to use a microprocessor means to adjust the applanation pressure determination for differences in corneal thickness and to record for the clinician an adjusted intraocular pressure.

It is a further object of the present invention to use an applanation component designed to allow the operator to atraumatically measure intraocular pressure without the requirement to view the corneal surface with a microscope at the point of applanation, thereby facilitating easy use of the device by minimally skilled personnel.

It is a further object of the present invention to use an applanation component designed to eliminate the requirement for use of a fluorescent dye on the cornea during applanation.

It is a further object of the present invention to use a non-visual means of obtaining the clinical endpoint.

It is a further object of the present invention to accurately measure intraocular pressure with the patient in any position.

Other objects and purposes for this invention will occur to those skilled in the art upon review of the invention as described and analyzed herein in light of its drawings and teachings. The present invention is not to be restricted in form nor limited in scope except by the claims appended hereto.

### BRIEF DESCRIPTION OF THE DRAWINGS

Referring to the drawings,

FIG. 1 is an illustration of the present invention showing a tonometery/pachymeter system handpiece in use on a human eye according to the present invention and providing a more accurate intraocular pressure determination;

FIG. 2 is a cross section of a first embodiment of a tonometer/pachymeter handpiece assembly having a pressure measurement means located proximal to the applanation surface and in functional relation to the cornea for determining uncorrected intra-ocular pressure. It is located concentrically within the distal end of the handpiece with an ultrasonic transducer and acoustic coupler for measuring corneal thickness;

FIG. 3 is a partial cross-section of a second embodiment of a tonometer/pachymeter handpiece and transducer assembly having a pressure measurement means in the distal end of the probe subjacent to an ultrasonic transducer assembly showing the ultrasonic transmission and reflection signals for determination of corneal thickness

FIG. 4A is a partial cross section of a transducer assembly similar to that shown in FIG. 2, highlighting the pressure measurement means which includes a displacement extension rod for transferring force from the cornea to a pressure transducer;

FIG. 4B is a preferred in body meant showing a partial cross section of a transducer assembly in accordance with the present invention, highlighting a fluid relaying mechanism for transferring force from the cornea to a pressure transducer and with an external pressure coupling membrane covering the cornea contact surface;

FIG. 5 is a cross section of the present invention having a displacement transducer for determining uncorrected intraocular pressure;

FIG. 6 is a partial cross-section of an embodiment of the tonometer/pachymeter transducer handpiece assembly having a corneal thickness measuring ultrasonic transducer assembly concentrically located in the distal end of the probe and having a pressure transducer subjacent and centrally positioned therein along with an eye-stabilizing fixation point and pushbutton actuator;

FIG. 7 is another embodiment of the present invention utilizing multiple corneal positioning sensors located within the corneal contact surface area of a tonometer/pachymeter transducer assembly;

FIG. 8 is a typical pressure measurement signal collected in accordance with the present invention; and

FIG. 9 is a typical ultrasound signal for cornea thickness determination collected in accordance with the present invention.

### DESCRIPTION OF THE PREFERRED EMBODIMENT

It is a preferred embodiment of the present invention to obtain more accurate intraocular pressure measurements using a solid-state, ultrasonic cornea thickness measuring means working in the 10 to 20 MHz frequency domain in functional association with a pressure sensing means as an applanation surface of predetermined area for contact with the corneal surface.

In another preferred embodiment, the applanation surface is a replaceable membrane.

In another preferred embodiment, the pressure sensing means is located proximal to the applanation surface and in functional relation to the corneal surface.

In another preferred embodiment, the device displays a digital LED readout of the applanation pressure, the corneal thickness and the intraocular pressure adjusted for corneal thickness.

It is yet a further preferred embodiment in which the measurement system incorporates a sensing means responsive to proper positioning of the system.

### EXAMPLE 1

A patient preparing for Laser Assisted In situ Keratomileusis (LASIK) photorefractive surgery for minus eight diopters (−8D) of myopia has a preoperative central corneal thickness of 452 microns. Six months following the LASIK procedure the intraocular pressure is measured as determined by Goldmann tonometery as 16 mmHg. The uncorrected intraocular pressure as determined by the present invention is also 16 mmHg. Pachymetry indicates the central corneal thickness to be 347 microns. The corrected intraocular pressure as determined by the present invention is 25 mmHg. In this example the present invention demonstrated that the intraocular pressure was higher than would be otherwise apparent; potentially masking glaucoma. The normal intraocular pressure ranges from 12 to 21 mmHg.

### EXAMPLE 2

A patient presented for a routine of found that examination has an intraocular pressure of 19 mmHg as determined by Goldmann tonometery. The uncorrected intraocular as determined by the present invention is also 19 mmHg. Pachymetry indicates the central corneal thickness to be 485 microns. The corrected intraocular pressure as determined by the present invention is 23 mmHg. In this example the present invention demonstrated that the intraocular pressure was higher than would be otherwise apparent; masking glaucoma.

The apparatus of this invention described and shown herein is a novel device for simultaneous measurement, at the same locus of applanation, pressure and surface thickness of a fluid filled sphere for more accurate determination of intracavity pressure, wherein at least a portion of the applanation surface is an ultrasonic transducer. The method for utilizing this device includes the simultaneous measurement, at the same locus of applanation, intracavity pressure and surface thickness of a fluid filled sphere for more accurate determination of intracavity pressure. In addition this novel device provides for simultaneous measurement, at the same locus of applanation, tonometery and pachymetry for determination of more accurate intraocular pressure, wherein at least a portion of the applanation surface is an ultrasonic transducer. Further, the method and device of the invention herein can provide for a fixation light source to stabilize the patient eye during applanation. Further yet, this invention includes a method of simultaneous measurement, at the same locus of applanation tonometery and pachymetry for the purpose of more accurate intraocular pressure determination. The locus of applanation tonometery and pachymetry is preferably the cornea of the eye.

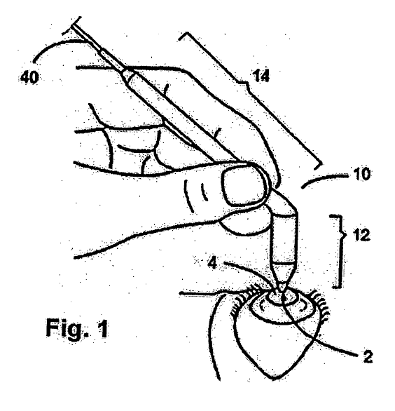

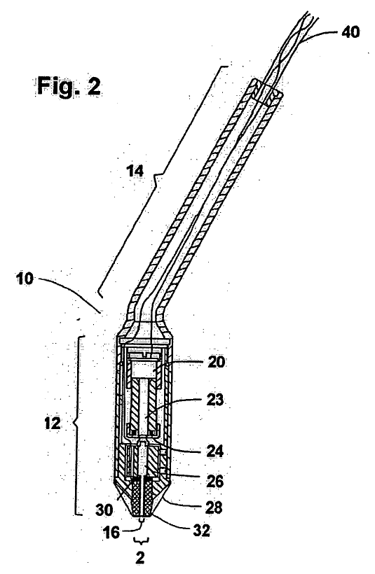

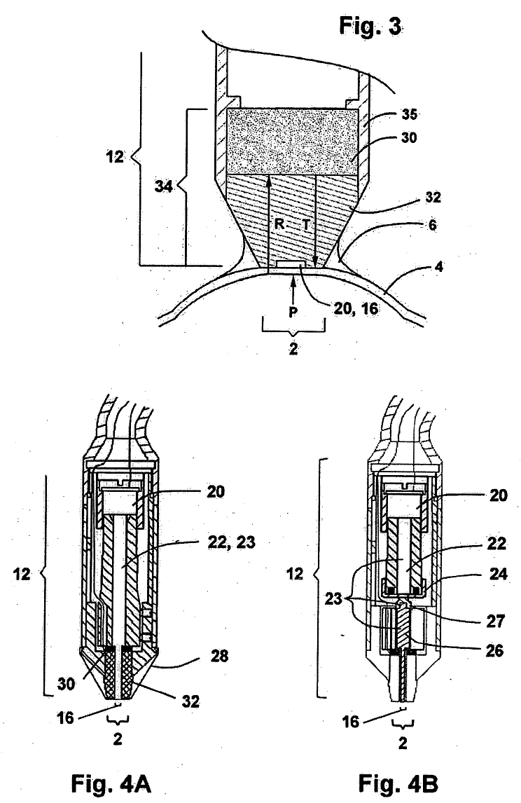

Referring now to the drawings, FIG. 1 illustrates a tonometer/pachymeter handpiece 10 suitable for contact by corneal contact surface 2 to cornea 4 and containing transducer assembly 12 and handpiece wand 14 according to an embodiment of the present invention. Tonometer/pachymeter transducer assembly 12 as shown in greater detail in FIG. 2 and FIG. 3 includes ultrasonic transducer assembly 33 and pressure transducer 20. Ultrasonic transducer assembly 33 is comprised of ultrasonic transducer crystal 30 and acoustic coupler 32 which can be made of any material suitable to transmit ultrasonic waves. Ultrasonic waves T are generated from ultrasonic transducer crystal 30 and transmitted or intensified through acoustic coupler 32. Ultrasonic waves R return to crystal 30 through acoustic coupler 32 following reflection or echo from distal surface of cornea 4. Ultrasonic transducer assembly 33 is held in position by outer housing 35. Force from cornea 4 is sensed by pressure transducer 20.

As shown in FIG. 4A and FIG. 4B, pressure transducer 20 may be proximal to cornea contact surface 2 wherein relay mechanism 23 is used to transfer pressure from cornea 4 to pressure transducer 20. Relay mechanism 23 may be air or other fluid 22 as shown in FIG. 4A or a solid material as shown in FIG. 4B. Relay mechanism 23 may be comprised of displacement extension rod 26, coupler 27 and fluid 22 or fluid 22 alone. Relay mechanism 23 may alternatively be displacement extension rod 26 coupled directly to pressure transducer 20. In the preferred embodiment relay mechanism 23 is air or other gaseous fluid, sealed to the environment through external pressure coupling membrane 28. External pressure coupling membrane 28 can also serve as a sterile barrier for contact with the cornea 4. It can also be used to seal relay mechanism 23.

As shown in FIG. 3 force transducer 20 may be embedded in and subjacent to acoustic coupler 32 in the distal end of assembly 12 and make up a portion of cornea contact surface 2. When cornea contact surface 2 of transducer assembly 12 is gently pressed or applanated and momentarily flattens cornea 4 to an area beyond pressure sensitive area 16, the only force sensed will be the intraocular pressure. If pressure sensitive area 16 is 3.06 mm in diameter, the measured IOP is the same as that from a Goldmann instrument without orbit furry corrections. It should be noted that while any size pressure sensitive area 16 can be used, the smaller the surface area the least traumatic for the patient.

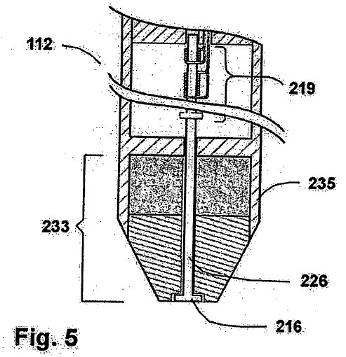

Alternatively as shown in FIG. 5 determination of IOP can be accomplished by use of displacement transducer 219 and displacement extension rod 226 that will generate a signal proportional to the indentation of pressure sensitive area 216. Cornea contact surface 2 creates an ultrasonic junction with cornea 4 that transmits ultrasonic transducer crystal 30 signals to and communicates reflected ultrasound signals from cornea 4. The ultrasonic signal reflected from the posterior surface of cornea 4 and communicated back through acoustic coupler 32 and detected by ultrasonic transducer crystal 30 is proportional to the thickness of the cornea. Transducer assembly 12 is preferably positioned at the geometric center of corneal cornea 4. Signal conditioning electronics and microprocessor (not shown) are programmed to receive output signals from ultrasonic transducer crystal 30 and pressure transducer 20 and display intraocular pressure measurements corrected for corneal thickness; the true intra-cavity pressure.

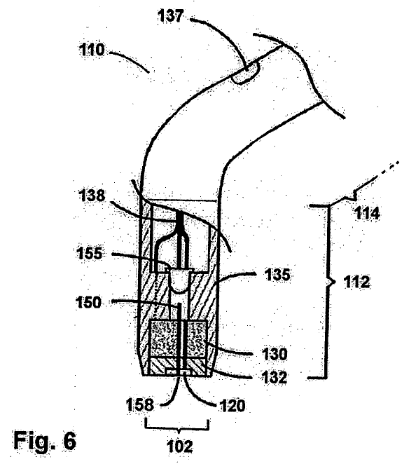

FIG. 6 illustrates another embodiment of the interior elements of tonometer/pachymeter handpiece 110 in accordance with the present invention. In this configuration contact surface 102 is formed from the tapered distal portion of outer jacket 135, acoustic coupler 132, pressure transducer 120 and fixation point 158. Fixation point 158 is shown as the distal end of optical coupler 150. Optical coupler 150 is shown as a short length of fiber optic but can be any other optical transmitting material or air. It is illuminated by a light emitting diode 155 or similarly functional illuminating device.

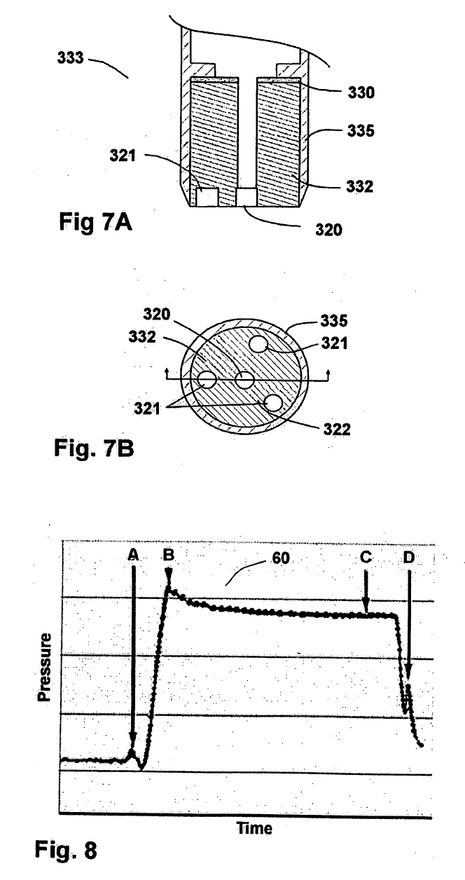

FIG. 7A and FIG. 7B show a cross-section and end view, respectively, of an ultrasonic transducer assembly 333 consistent with the teaching of the invention in which multiple cornea positioning transducer 321 are shown. In the illustrated embodiment, three cornea positioning transducer 321 are concentrically located 120° around pressure transducer 20. However, positioning transducer's 321 can be any distal location provided they are selected to be responsive to contact with cornea 4. In this configuration signals can be produced indicating that cornea contact surface 2 is uniformly and perpendicularly in contact with cornea 4.

FIG. 8 is data representative of a typical pressure measurement signal generated using the configuration shown in FIG. 4A where pressure signal 60 is a pressure versus time tracing of pressure exerted on pressure transducer 20 resulting from applanation of cornea contact surface 2 on cornea 4. Pressure signal 60 at time ‘A’ represents initial depression of cornea contact surface 2 to cornea 4. ‘B’ represents a signal overshoot, ‘C’ represents true applanation pressure not corrected for thickness of cornea 4 and ‘D’ represents buckling of cornea 4 resulting from excessive force on cornea contact surface 2. Signal conditioning electronics (not shown) assess the data representative of pressure measurements and extracts and display true intraocular pressure ‘C’.

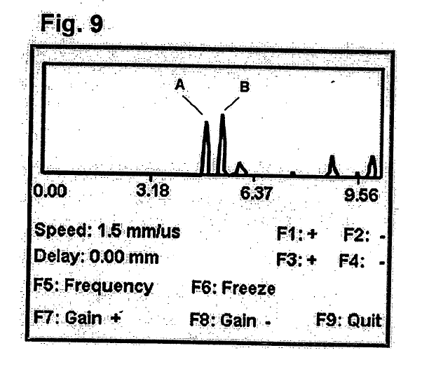

FIG. 9 is data representative of ultrasonic waves generated by ultrasonic transducer crystal 30 and reflecting from cornea contact surface 2 (signal ‘T’ in FIG. 3) and shown as peak intensity ‘A’ and ultrasonic waves reflecting from the distal surface of cornea 4 (signal ‘R’ in FIG. 3) and shown as peak intensity ‘B’. Time difference between peak intensity ‘A’ and ‘B’ is proportional to thickness of cornea 4.

Variations or modifications to the subject matter of this invention may occur to those skilled in the art upon review of the summary provided herein, in addition to the description of its preferred embodiment, in light of the drawings. Such variations, if within the spirit of this invention, are intended to be encompassed within the scope of the invention as described herein.

1. An applanation apparatus which allows for the determination of applanation pressure and membrane thickness of a sphere having a fluid filled cavity comprising: an applanation surface incorporating an area for generation of a signal responsive to pressure, an ultrasonic transmitter and receiver for generation of a signal responsive to membrane thickness and in functional relationship with said applanation surface, a signal processor utilizing said pressure signal and said ultrasonic signal in the determination of intracavity pressure and membrane thickness of the sphere.

2. The apparatus of claim 1 wherein said signal processor is a microprocessor to determine intracavity pressure of a sphere independent of the thickness of said membrane thickness.

3. The apparatus of claim 1 wherein said applanation apparatus determines intracavity pressure and membrane thickness at the same locus.

4. The apparatus of claim 1 wherein said applanation surface incorporating an area responsive to pressure is a piezo-resistive sensor.

5. The apparatus of claim 1 wherein said ultrasonic transmitter and receiver are the same device.

6. The apparatus of claim 1 wherein said area responsive to pressure is coaxial with said ultrasonic transmitter and receiver.

7. The area responsive to pressure of claim 6 is subjacent with said applanation surface.

8. The apparatus in claim 1 wherein a pressure coupling membrane is positioned over the applanation surface.

9. The coupling membrane in claim 8 wherein said membrane is replaceable.

10. The area responsive to pressure in claim 1 wherein a solid state pressure transducer is in functional relationship to said sphere through a relay mechanism.

11. The relay mechanism in claim 10 is a fluid.

12. The fluid in claim 11 is air.

13. The relay mechanism in claim 10 is a solid rod.

14. The relay mechanism in claim 10 comprising any combination of relay mechanisms in claims 11 and 13.

15. The apparatus of claim 1 wherein one or more position indicators signal proper contact with said sphere

16. The position indicator in claim 15 is comprised of geometrically positioned transducers.

17. The position indications in claim 15 are force transducers.

18. An applanation apparatus which allows for the determination of intracavity pressure and membrane thickness of the eye comprising: an applanation surface incorporating an area for generation of a signal responsive to pressure, an ultrasonic transmitter and receiver for generation of a signal responsive to membrane thickness and in functional relationship with said applanation surface, a signal processor utilizing said pressure signal and ultrasonic signal in the determination of intracavity pressure adjusted for membrane thickness of the eye.

19. The apparatus of claim 18 wherein the membrane thickness of the eye is the thickness of the cornea.

20. The apparatus of claim 18 wherein said signal processor is a microprocessor to determine intracavity pressure independent of corneal membrane thickness.

21. The apparatus of claim 18 wherein said applanation apparatus determines intracavity pressure and membrane thickness at the same locus.

22. The apparatus of claim 18 wherein said area for generation of a signal responsive to pressure is a piezo-resistive sensor.

23. The apparatus of claim 18 wherein said ultrasonic transmitter and receiver are the same device.

24. The apparatus of claim 18 wherein said applanation surface incorporating an area responsive to pressure is coaxial with said ultrasonic transmitter and receiver.

25. The apparatus of claim 23 wherein said ultrasonic transmitter and receiver is subjacent to said applanation surface.

26. The applanation apparatus of claim 18 wherein said applanation apparatus includes a fixation point.

27. The apparatus in claim 18 wherein a pressure coupling membrane is positioned over the applanation surface.

28. The coupling membrane in claim 27 is sterile.

29. The coupling membrane in claim 27 is replaceable.

30. A method of determining intracavity pressure of an eye comprising:
placement of an applanation surface against a cornea incorporating a pachymeter and tonometer transducer and maintaining contact with the cornea while measuring cornea thickness and intracavity pressure.

31. The method of claim 30 wherein measurement of cornea thickness and intracavity pressure includes placing a pressure coupling membrane over said applanation surface.

32. The method of claim 30 wherein determination of intracavity pressure is accomplished without the necessity to view the cornea through a slit-lamp microscope.

33. The method of determining intracavity pressure of claim 30 wherein placing said pachymeter and tonometer transducer is positioned by hand.

34. The method of claim 30 wherein a position indicator signals proper contact of said transducer with the cornea.

35. An applanation apparatus which allows for the determination of applanation pressure and membrane thickness of a human eye having a fluid filled cavity comprising:
an applanation surface incorporating an area responsive to pressure, an ultrasonic transmitter and receiver in functional coaxial relationship with said applanation surface for sending and receiving an ultrasonic signal to said applanated cornea, said ultrasonic signal and pressure signal processed to determine the applanation pressure and membrane thickness of the human eye.

36. The applanation apparatus of claim 35 including a microprocessor, the microprocessor capable of receiving said ultrasonic signal and said pressure signal, said ultrasonic signal being indicative of corneal membrane thickness and said applanation pressure signal being indicative of intracavity pressure, said microprocessor capable of correcting said pressure signal for a corneal membrane thickness as determined by said ultrasonic signal and to determine a true intracavity pressure independent of corneal membrane thickness.

37. The applanation apparatus of claim 36 wherein said microprocessor determines said membrane thickness and said intracavity pressure at the point of applanation.

38. The applanation apparatus of claim 35 wherein said applanation surface area responsive to pressure is a piezoresistive sensor.

39. The applanation apparatus of claim 35 wherein said ultrasonic transmitter and receiver are the same device.

40. The applanation apparatus of claim 35 wherein said ultrasonic transmitter and receiver is in a coaxial relationship with said applanation surface area responsive to pressure.

41. The applanation apparatus of claim 40 wherein said ultrasonic transmitter and receiver is within said applanation surface.

42. The applanation apparatus of claim 35 wherein said applanation apparatus including a fixation point is coaxially positioned within said applanation surface.

43. A method of determining intraocular pressure of a human eye comprising: placing an applanation apparatus against a cornea, said apparatus including a corneal contact surface for placing against the cornea, an ultrasonic transmitter and receiver within said body, a pressure sensing means, said ultrasonic transmitter and receiver and said pressure sensing means being in communications with a microprocessor, said microprocessor being capable of correcting a pressure signal for a corneal membrane thickness and to determine a true intraocular pressure independent of corneal membrane thickness, creating an applanation point on the cornea with said corneal contact surface, measuring the membrane applanation pressure at said applanation, measuring membrane thickness with said ultrasonic transmitter and receiver; and correcting the measured intracavity pressure for membrane thickness.

44. The method of determining intraocular pressure of claim 43 without the requirement of viewing the cornea though a slit-lamp microscope.
